(19) United States
(12) Patent Application Publication
(10) Pub. No.: US 2011/0190740 A1
(43) Pub. Date: Aug. 4, 2011

(54) PLACIDO RING MEASUREMENT OF ASTIGMATISM AXIS AND LASER MARKING OF ASTIGMATISM AXIS

(75) Inventors: Rudolph W. Frey, Winter Park, FL (US); Steven E. Bott, Oviedo, FL (US)

(73) Assignee: LensAr, Inc.

(21) Appl. No.: 13/017,499

(22) Filed: Jan. 31, 2011

Related U.S. Application Data
(60) Provisional application No. 61/300,129, filed on Feb. 1, 2010.

(51) Int. Cl. A61B 18/20; A61B 3/107

(52) U.S. Cl. 606/5; 351/212; 351/246

(57) ABSTRACT

A laser treatment system that includes means for measuring an astigmatism axis of a cornea of an eye of a patient and means for applying a laser beam to the eye.

This application claims the benefit of priority under 35 U.S.C. §119(e)(1) of U.S. Provisional Application Ser. No. 61/300,129 titled Placido Ring Measurement of Astigmatism Axis and Laser Marking of Astigmatism Axis, filed Feb. 1, 2010, the entire contents of which is incorporated herein by reference.

### FIELD OF THE INVENTION

The present invention relates to a system for performing an astigmatism measurement for the purpose of correcting astigmatism. The present invention also has to do with marking the measured axis of astigmatism with a laser-created mark.

### BACKGROUND

In known procedures for correcting astigmatism, such as limbal relaxing incisions, LASIK or implantation of toric IOLs, it is important to register the respective treatment or device in precise alignment relative to the eye's axis of astigmatism. While each procedure is performed while the patient is in a reclining position, the prior astigmatism measurement is conventionally made with the patient in a sitting position. During the process of the patient being moved from the sitting position to the reclining position, cyclotorsion (rotation of the eye about its optical axis) generally occurs. Accordingly, it is necessary to correct for cyclotorsion prior to performing the procedure. Cyclotorsional compensation is generally performed by making a registration mark on the eye, using an ink marker, while the patient is in a sitting position. The registration marker is used when the patient is in the reclining, treatment position to adjust for any rotation of the axis of astigmatism which might occur.

The use of ink marks reduces the effect of cyclotorsion on the astigmatism treatment; however, it is inconvenient—it requires a separate seating of the patient at a slit lamp—and has limited accuracy because of the inevitable errors in manually placing the initial marks, and the “bleeding” of the marks as the tear film reacts with the marking ink.

### BRIEF SUMMARY

One aspect of the present invention regards a laser treatment system that includes means for measuring an astigmatism axis of a cornea of an eye of a patient and means for applying a laser beam to the eye after the means for measuring has measured the astigmatism axis.

A second aspect of the present invention regards an astigmatism axis measurement system that includes means for directing light toward a cornea of an eye of a patient and means for measuring an astigmatism axis of the cornea based on light reflected off of the cornea.

A third aspect of the present invention regards an astigmatism axis measurement system that includes means for measuring an astigmatism axis of a cornea of an eye of a patient and means for determining an apex of the cornea.

A fourth aspect of the present invention regards an astigmatism axis measurement system that includes an annular source generating a light beam that is directed toward a cornea of an eye of a patient and a detector for receiving light reflected from the cornea. A processor for receiving signals from the detector and determining an astigmatism axis of the cornea.

A fifth aspect of the present invention regards a method of identifying an astigmatism axis of a cornea or an eye of a patient, the method including directing annular light beams toward a cornea of an eye of a patient and receiving light reflected from the cornea. The method further including determining an astigmatism axis of the cornea based on the received light.

A sixth aspect of the present invention regards a method of marking an eye as to where an astigmatism axis of a cornea exists, the method including determining an astigmatism axis of the cornea and marking the eye with a laser beam so as to form a tag on the eye that identifies the astigmatism axis.

A seventh aspect of the present invention regards a laser treatment system that includes means for generating light beams directed toward a cornea of an eye of a patient and means for receiving light reflected from the cornea. The system further including means for determining a shape of the cornea based on the received light and means of applying a laser beam to the eye after the means for determining the shape of the cornea has determined the shape of the cornea.

An eighth aspect of the present invention regards a laser treatment system that includes multiple annular sources generating light beams directed toward a cornea of an eye of a patient and a detector for receiving light reflected from the cornea. The system further including a processor for receiving signals from the detector and determining a shape of the cornea based on the received light. The system includes a laser that applies a laser beam to the eye based on the shape of the cornea determined by the processor.

A ninth aspect of the present invention regards a method of measuring a corneal shape that includes generating light beams directed toward a cornea of an eye of a patient and receiving light reflected from the cornea. The method further including determining a shape of the cornea based on the received light and applying a laser beam to the eye based on the determined shape of the cornea.

One or more aspects of the present invention allow for a quick registration and immobilization of an eye.

### BRIEF DESCRIPTION OF THE DRAWING

The accompanying drawings, which are incorporated herein and constitute part of this specification, and, together with the general description given above and the detailed description given below, serve to explain features of the present invention. In the drawings:

FIG. 1 schematically shows an embodiment of a measuring system for measuring the corneal astigmatism axis prior to an ophthalmological procedure being performed on the eye of a patient in accordance with the present invention;

FIG. 2 schematically shows operation of an embodiment of a telecentric detection system for placido ring measurements that is used with the measuring system of FIG. 1 in accordance with the present invention;

FIG. 3 shows picture of a common toric intraocular lens (IOL) implanted in an eye after the corneal astigmatism axis of the eye has been determined and marked using a treatment laser, using the measuring system of FIG. 1 in accordance with the present invention; and

FIG. 4 schematically shows laser cut capsulotomy openings in the anterior crystalline lens capsule cut with a “tag” to mark the axis of astigmatism that is measured by the measuring system of FIG. 1 in accordance with the present invention.

### DETAILED DESCRIPTION OF THE PREFERRED EMBODIMENTS

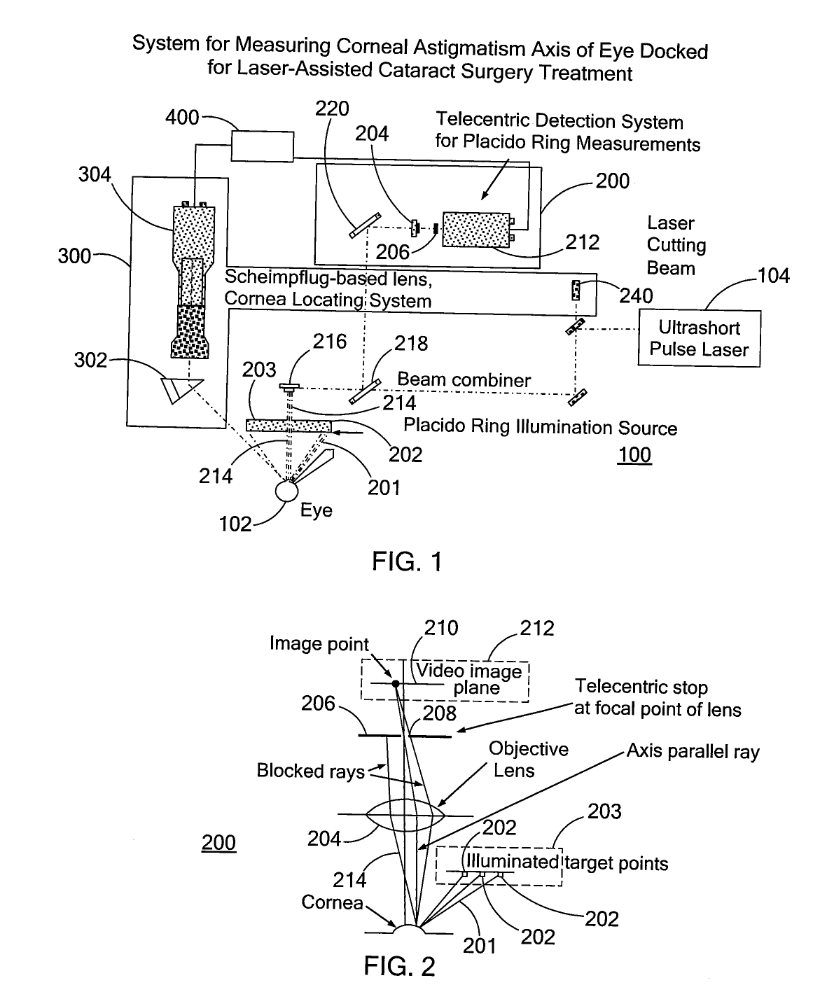

FIG. 1 schematically shows a measuring and treatment system 100 for measuring the corneal astigmatism axis and for performing an ophthalmological procedure on the eye 102 of a patient. The system 100 includes a telecentric detection system 200 for Placido Ring measurements, a Scheimpflug-based lens and cornea locating system 300, and a treatment laser system that includes a treatment laser 104.

In use, the patient is typically lying on a gurney or reclining surgical chair which is rolled into position under the optical head of the treatment laser 104. The telecentric detection system 200 and the Scheimpflug-based lens and cornea locating system 300 may be designed to work with the patient in a reclining position under the treatment laser system since in this position the cyclotorsion of the eye, which occurs when a patient who is in a sitting position (for example to allow conventional astigmatism measurements to be made) changes to a reclining position, has already occurred. It is also advantageous that the Placido Ring detection system 200 and the Scheimpflug-based lens and cornea locating system 300 are so located such that the patient can remain stationary for both the measurements and laser treatment, since this obviates or lessens the time consuming step of re-aligning the patient with the laser for the subsequent laser treatment.

After the corneal astigmatism axis is found using the Placido Ring-based measurement alone or the Placido ring-based measurement enhanced by inclusion of a measurement of the position of the corneal apex by a separate independent measurement system, a medical procedure can be performed with the laser systems described in U.S. patents applications Ser. Nos. 11/337,127; 12/217,285; 12/217,295; 12/509,412; 12/509,021; 12/509,211 and 12/509,454, the entire contents of each of which are incorporated herein by reference. Possible procedures to be performed by the laser systems to correct or reduce astigmatism are the performance of limbal relaxing incisions or LASIK. Another possible procedure is the use of the treatment laser to assist in cataract removal and the subsequent implantation of a toric or other IOL.

Operation of measuring system 100 includes having the patient lie on a patient bed in position for the laser surgery. Next, the optical head of the treatment laser 104 is aligned, using a joystick that controls a 3-axis motion control system, to the patient's cornea. The optical head of the treatment laser system houses both the Placido Ring detection system 200 and the Scheimpflug-based lens and cornea locating system 300 as well as the optics that are used to guide the treatment laser beam. Thus, aligning this optical head relative to the patient serves the purpose of aligning all three systems (200; 300 and treatment laser system) simultaneously relative to the patient's eye and, thus, reduces the need for time consuming re-alignments for the sequential operations. A sensor, not shown, detects when the z position (position along a direction parallel to the axis of the laser beam passing through a Placido Ring light generator 203 as shown in FIG. 1) is correct for the astigmatism axis measurement; the sensor generates a signal when the eye is at the correct distance below the Placido Ring light generator 203. A camera system and display monitor, not shown, positioned above generator 203 and staring directly downward at the eye through the center of light generator 203, provides an image of the eye, viewed from above, to allow the x, y position of the optical head of the treatment laser 104 to be centered on the eye. A software reticule is superimposed on the image of the eye on the camera's monitor, to assist in the assessment of centration.

After the z-position for the optical head of the treatment laser 104 is determined, and the Placido Ring light generator 203 is centered directly above the eye then measurement of the astigmatism axis is performed using telecentric detection system 200 for Placido Ring measurements. Operation of system 200 is understood upon a review of FIG. 2. As shown, light 201 from one or more concentric ring sources 202 of light generator 203 is directed towards the cornea of the eye 102 and then reflected light 214 is directed towards an objective lens 204. Note that the ring sources 202 are concentric relative to an axis of the treatment laser beam passing through the opening of the light generator 203. In addition, a ring source can be a single light element in the shape of a circle or multiple, discrete light elements positioned on a circle.

Next, the light from objective lens 204 is directed through a telecentric stop 206 that is positioned at a focal plane of the lens 204. The stop 206 includes an opening 208 positioned at a focal point of the lens 204 so that only light reflected from the cornea that was initially parallel to the axis of the objective lens is allowed to pass through the opening 208 and be received on the video image plane 210 of a detector 212. As shown in FIG. 1, additional optics, such as a beam scanning system 216, beam combiner 218 and beam splitter 220, can be used to direct the reflected light 214 toward the lens 204.

Applying the above principles to detection system 200, one or more concentric (relative to the axis of laser beam from optical head 104, which is collinear with the axis of the objective lens, 204, in FIG. 2) diverging beams of light 201 are directed from the annular sources 202 toward the cornea of the eye 102. If the cornea were perfectly spherical in shape, then the beams of light 201 which reflect from the cornea into a direction parallel to that of the objective lens 204 would pass through the telecentric stop aperture 208 and form concentric circles of light on the video image plane 210. If the cornea is astigmatic, its shape will deviate slightly from that of a perfect sphere in such a way as to cause the image of the reflection of the Placido Ring illumination sources to have a nearly elliptical shape. Measurement of the shape of the nearly elliptical images formed on the video image plane 210, using standard numerical methods such as those described in Turuwhenua, Jason, “An Improved Low Order Method for Corneal Reconstruction”, Optometry and Vision Science, Vol. 85, No. 3, March 2008, pp. E211-E218, the shape of the cornea can be determined by a processor. Note that the overall shape of the cornea could be measured if multiple annular sources 202, such as 12 to 24 in number, are used. Alternatively, if only the axis of the astigmatism is desired, similar methods can be used to numerically extract the axis of the astigmatism from the shapes of the reflected light images of the Placido Ring light generator 203 on the video image plane 210. If only the astigmatism axis is needed, a simple method of extracting it from the reflected images is to determine the angles of the semi-major axes of the ellipses using a simple least squares curve fitting technique.

Note that it is possible that de-centration errors may occur if the system 200 is used in isolation. De-centration of the Placido rings from a position concentric with the corneal apex would be a major source of inaccuracy in measurement of the axis of astigmatism. In order to correct for this, a simultaneous or nearly simultaneous measurement of the corneal apex made by the Scheimpflug system 300 with the measurement of system 200 is used to correct the astigmatism axis measurement made by system 200 for de-centration errors. As shown in FIG. 1, a super luminescent diode (SLD) 240 projects a beam of light toward a beam scanning system 216 which in turn projects the beam of light onto the eye. The beam scanning system 216 is controlled by a computer. The scanning system 216 sequentially scans a light beam so as to create a “sheet” of light through the eye; the “sheet” contains the primary axis of the beam scanner 216 and is perpendicular to the plane of the page of FIG. 1. The light scattered by the anterior cornea surface from the sheet of light is reflected by the prism 302 into a camera 304, which stores an image of the scattering from each scanned line. The process is repeated for one or more additional “sheets” of light, each parallel to the first and displaced to the left or right of the first. The camera 304 image of each “sheet” of light forms a longitudinal section of the eye, with the position of the cornea appearing as a bright arc at the top of eye. Each longitudinal section is displaced from the others. They might be visualized as sections of egg sectioned with an egg cutter.

The camera 304 is then repositioned to a point, out of the plane of the page of FIG. 1, such that the plane defined by the camera lens axis and the primary axis of the beam scanning system 216 is perpendicular to the plane of the page of FIG. 1. In this second camera position, the foregoing process is repeated, with the scanned “sheets” of light now being parallel to the plane of the page of FIG. 1.

The light scattered from the nearly spherical anterior corneal surface form circular images on the camera 304. Using ray tracing, the longitudinal sections from the camera images can be used to find the location of the cornea, relative to the system 300 and also to system 200 and the treatment laser since their positions are known relative to each other, along the corneal arc of each longitudinal section. By fitting these various arcs to a spherical shape, using simple least squares fit, the position of the overall cornea can be found and the position of the corneal apex directly derived.

After the Placido ring measurements previously described are made by systems 200 and 300, the optical head of treatment laser 104 is moved directly upward, out of the way, to allow access to the patient's eye 102 for application of a suction ring. In operation, a suction ring (not shown) is applied manually to the patient's eye 102. After the suction ring is applied, the optical head of treatment laser 104 is docked, using the previously described joystick. Since the patient's eye 102 has not been moved and since the treatment laser 104 and the astigmatism measuring systems 200 and 300 are aligned to each other, the treatment laser 104 can now be used to correct or reduce the astigmatism of the eye 102, based on the previously described astigmatism axis determination and/or the corneal shape determination, using limbal relaxing incisions or LASIK, aligning the astigmatism treatment to the measured axis of astigmatism.

The above described alignment system and process can also be applied to procedures that involve implanting a toric intraocular lens (IOL) to treat astigmatism. Note that IOLs are synthetic lenses implanted into the capsular bag in the eye, after a cataractous lens is removed. The IOL restores vision by replacing partially opaque cataratous lens with a clear lens of appropriate power. A conventional IOL has only spherical power. A toric IOL has both spherical and cylindrical power and can thus correct astigmatism in the eye.

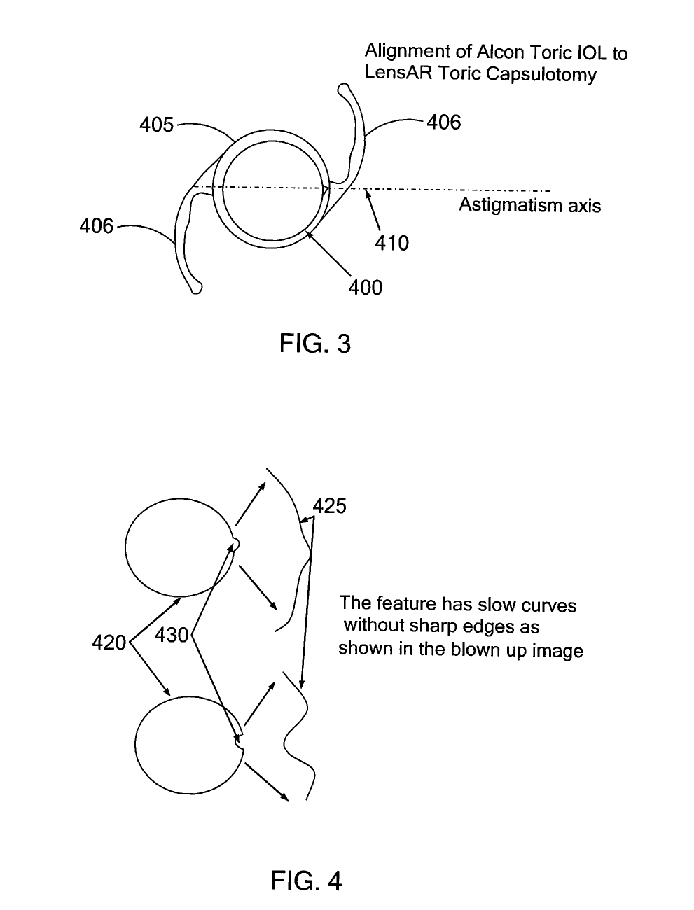

In the case when a toric IOL is to be subsequently implanted to treat astigmatism, the treatment laser 104 can be used to mark the axis of astigmatism for later use in aligning the axis of astigmatism 410 (shown in FIG. 3) of the IOL 405 (with haptics 406 used for anchoring IOL 405 in the capsular bag), with the marked axis of astigmatism of the eye 102. In cataract procedures, a round opening is manually torn or cut by a laser in the crystalline lens anterior capsule. The cataractous lens is removed through the opening and an IOL is placed into the capsular bag, generally centered behind the capsular opening. The treatment laser 104 can be used to cut a small “tag” as part of the circular capsulotomy 400. The “tag” provides a visible reference mark along which the axis of astigmatism of the IOL 410 can be aligned. As shown in FIG. 4, the “tags” 430 in the capsular openings can be positioned inwardly or outwardly. The “tag” is cut in a smooth curve along the capsulotomy cut to avoid risk of radial capsular tears during the cataract procedure. Possible smooth shapes of the “tags” are shown schematically in close-up 425. This method of marking the astigmatism axis by incorporating a “tag” in the capsulotomy allows the astigmatism mark, i.e. the “tag” to be ideally placed for use in aligning the astigmatism axis of the IOL. The “tag” is in the immediate vicinity of the astigmatism mark on the IOL and may in fact be directly over the astigmatism axis mark on the IOL, avoiding any errors in registration which might occur when aligning the IOL mark with, for example, an ink mark on the sclera, a considerable distance from the IOL. In summary, the “tag” provides a visual marker so that the surgeon implanting a toric IOL can line up the astigmatism axis of the IOL with marked axis of astigmatism of the eye 102.

To avoid any possible distortion of the astigmatism axis of the eye 102 which might occur when the a suction ring is placed on the eye 102 for docking with the optical head of the treatment laser 104, a small mark, for example a line, could be made by the laser in the center of the lens capsule immediately after the astigmatism axis was measured as described above. Then, after affixing the suction ring and docking the eye 102 to the optical head, the marks in the center of the capsule could be used, either manually or using automatic image recognition techniques built into a computer program, to set the position of the “tag”—marked laser-cut capsulotomy for use in the toric IOL implantation.

Still another alternate method of marking the astigmatism axis with the treatment laser would entail shooting several laser shots, either at full or reduced energy at the position of the astigmatism axis at the limbus to make a persistent visible reference mark.

Since the x, y position of the optical head of the treatment laser 104 is pre-aligned during the astigmatism axis measurement step, very little adjustment is needed to dock the optical head to the suction ring. Note that the telecentric viewing system 200 is also used as a general viewing system, to assist the laser system associated with the optical head of the treatment laser 104 when the optical head is docked to the suction ring.

Use of the measuring system 100 with the above described laser systems is advantageous. For example, the measuring system 100 would allow measuring the astigmatism axis in situ, while the patient is lying on the treatment bed, just in advance of the laser treatment—thus eliminating the need for pre-operative eye marks. In the case of performing limbal relaxing incisions, the automatic measurement of the astigmatism axis by system 100 increases the accuracy of the placement of the limbal relaxing incisions, thereby improving the efficacy of the treatment. The method can also be used in conjunction with the laser to mark the astigmatism axis for cyclotorsional registration of a toric IOL.

Despite the benefits of the method in convenience and more accurate, automatic placement of the treatment axis for astigmatism, there is no laser astigmatism treatment device which currently incorporates an astigmatism measuring system into the device. The present invention eliminates the need for manually marking the eye and circumvents the inaccuracies inherent in manual placing of marks as well as the dispersion of the ink marks by the eye's tear film. The integral astigmatism measurement, in combination with use of marks made by the treatment laser can be used to mark the axis of astigmatism for later registration of a toric IOL or for any subsequent refractive treatment of the eye requiring knowledge of the axis of astigmatism.

Since the measuring device is built into the optical head of the treatment laser 104, the alignment of the measuring 100 to the eye 102 reduces the time needed later to align the eye to the laser treatment system. The system 100 also makes dual use of a camera 212 and ring light sources 202 for both the astigmatism measurement and for general viewing of the eye during the eye docking and lasing parts of the procedure.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A laser system comprising:
means for measuring an astigmatism axis of a cornea of an eye of a patient; and
means for applying a laser beam to said eye after said means for measuring has measured said astigmatism axis.

2. The laser system of claim 1, wherein said means for applying a laser beam to said eye treats said eye.

3. The laser system of claim 1, wherein said means for applying a laser beam to said eye marks said eye so as to identify said astigmatism axis.

4. An astigmatism axis measurement system comprising:
means for directing light toward a cornea of an eye of an eye of a patient; and
means for measuring an astigmatism axis of said cornea based on light reflected off of said cornea.

5. An astigmatism axis measurement system comprising:
means for measuring an astigmatism axis of a cornea of an eye of a patient; and
means for determining an apex of said cornea.

6. An astigmatism axis measurement system comprising:
an annular source generating a light beam directed toward a cornea of an eye of a patient;
a detector for receiving light reflected from said cornea;
a processor for receiving signals from said detector and determining an astigmatism axis of said cornea.

7. The system of claim 6, further comprising a second detector that receives further light reflected from said cornea and determines an apex of said cornea.

8. A method of identifying an astigmatism axis of a cornea or an eye of a patient, the method comprising:
directing annular light beams toward a cornea of an eye of a patient;
receiving light reflected from said cornea; and
determining an astigmatism axis of said cornea based on said received light.

9. The method of claim 8, further comprising marking said eye with a laser beam so as to form a tag on said eye that identifies said astigmatism axis.

10. A method of marking an eye as to where an astigmatism axis of a cornea exists, the method comprising:
determining an astigmatism axis of said cornea; and
marking said eye with a laser beam so as to form a tag on said eye that identifies said astigmatism axis.

11. A laser treatment system comprising:
means for generating light beams directed toward a cornea of an eye of a patient;
means for receiving light reflected from said cornea;
means for determining a shape of said cornea based on said received light; and
means for applying a laser beam to said eye after said means for determining said shape of said cornea has determined said shape of said cornea.

12. A laser treatment system comprising:
multiple annular sources generating light beams directed toward a cornea of an eye of a patient;
a detector for receiving light reflected from said cornea;
a processor for receiving signals from said detector and determining a shape of said cornea based on said received light; and
a laser that applies a laser beam to said eye based on said shape of said cornea determined by said processor.

13. A method of measuring a corneal shape comprising:
generating light beams directed toward a cornea of an eye of a patient;
receiving light reflected from said cornea;
determining a shape of said cornea based on said received light; and
applying a laser beam to said eye based on said determined shape of said cornea.
